(19) United States
(12) Patent Application Publication
(10) Pub. No.: US 2015/0320334 A1
(43) Pub. Date: Nov. 12, 2015

(54) SYSTEM, METHOD AND COMPUTER-ACCESSIBLE MEDIUM FOR IMPROVING PATIENT COMPLIANCE DURING MAGNETIC RESONANCE IMAGING EXAMINATIONS

(71) Applicant: NEW YORK UNIVERSITY, New York, NY (US)

(72) Inventor: Kai Tobias Block, Brooklyn, NY (US)

(73) Assignee: New York University

(21) Appl. No.: 14/704,190

(22) Filed: May 5, 2015

Related U.S. Application Data
(60) Provisional application No. 61/989,021, filed on May 6, 2014.

(51) Int. Cl. A61B 5/055; G01R 33/28; H05B 33/08

(52) U.S. Cl. CPC A61B 5/055; A61B 5/0555; H05B 33/0854; G01R 33/283

(57) ABSTRACT

An exemplary system, method and computer-accessible medium can be provided for alerting a patient of a condition(s) during an MRI scan procedure(s), which can include, for example receiving information related to the condition(s), and controlling an intensity of a light source(s) located near or on an MRI scanner based on the condition(s). The light source(s) can include a light-emitting diode, which can be a white LED and/or a rainbow LED. The color of the rainbow LED can be changed based on the condition(s). The light source(s) can be located inside or outside a bore of the MRI scanner.

### CROSS-REFERENCE TO RELATED APPLICATION(S)

This application relates to and claims priority from U.S. Patent Application No. 61/989,021, filed on May 6, 2014, the entire disclosure of which is incorporated herein by reference.

### FIELD OF THE DISCLOSURE

The present disclosure relates generally to a magnetic resonance imaging (“MRI”), and more specifically, to exemplary embodiments of exemplary systems, methods, computer-accessible mediums and apparatus for improving patient compliance during MRI examinations using, for example, a scanner-controlled adjustment of lighting.

### BACKGROUND INFORMATION

Medical examinations using MRI can be highly sensitive to motion, and can benefit from strict immobility of the patient during the data acquisition. However, in clinical practice, sufficient suppression of movements may not typically be achieved, which can lead to severe motion artifacts in the obtained images. Often, the images can be rendered completely non-diagnostic. In these cases, examinations need to be repeated, potentially on a different day if an intravenous contrast agent has already been injected into the patient. Non-diagnostic examinations due to motion artifacts, therefore, can result in high costs, and can potentially lead to clinical misinterpretation if a scan cannot be repeated, and/or if important pathologic structures may not be visible on the motion-affected images. Overall, the high sensitivity of MRI to motion has resulted in limited acceptance of MRI as imaging modality of choice for certain clinical indications, including abdominal examinations or pediatric examinations, and has led to the preference of CT imaging for many applications

In most clinical MRI examination protocols, the patient is instructed, prior to the examination, to stop moving from the moment when the patient table is positioned in the center of the MRI magnet. Some MRI systems can facilitate the technicians to communicate with the patient during scan pauses using an intercom system (e.g., via earphones). Thus, it can be possible to remind the patient, in-between the scans, to suppress their movement. For specific examinations, such as abdominal examinations or cardiac examinations, it can be beneficial to suspend respiration during multiple scan periods of typically 15-20 seconds (e.g., breath-hold scans). For these examinations, the technicians, using the intercom, instruct the patient, prior to the data acquisition, to hold their breath, with a series of breath-hold commands, for example, “breathe out, breathe in, breathe out, stop breathing”.

Despite these existing measures to achieve patient compliance, patient motion remains a major problem in routine practice, and the volume of motion-affected studies with compromised diagnostic quality is high. Several reasons for this include, for example:
1. In many imaging centers with busy scheduling, technicians don't have the time to appropriately brief the patient and to communicate with the patient in between the individual scans.
2. Many patients can be surprised or frightened by the loud noise when the scan starts and, thus, they move suddenly because they are unaware of the timing of the MRI exam.
3. Many patients are unable to hear instructions given by the technicians due to impaired hearing capabilities, technical issues or improper setup of the intercom system which often uses headphones driven by air pressure through a long tube, or through an inability to understand a foreign language.
4. MRI examinations can be long, and last up to 90 minutes of duration or even longer. Patients often have the need to move for a short moment, but are unaware during what time periods they can move or when the next scan is about to start.
5. In breath-hold examinations, patients can be unaware of the exact timing of breath-hold commands. The speed of these commands can vary significantly among technicians. Sometimes, the speed of the breath-hold commands does not match to the normal respiration cycle of the patient.
6. Many patients can be scarred during the examinations, and can be unaware that their cooperation is needed. Also, they can be unaware when the examination is finished and when the patient table is about to be moved

Thus, it may be beneficial to provide an exemplary system, method, computer-accessible medium and apparatus that can facilitate easy communication with patients during an MRI scan, and which can overcome at least some of the deficiencies described herein above.

### SUMMARY OF EXEMPLARY EMBODIMENTS

These and other objects of the present disclosure can be achieved by provision of an exemplary system, method, computer-accessible medium and apparatus for alerting a patient of a condition(s) during an MRI scan procedure(s), which can include, for example receiving information related to the condition(s), and controlling an intensity of a light source(s) located near or on an MRI scanner based on the condition(s). The light source(s) can include a light-emitting diode, which can be a white LED and/or a rainbow LED. The color of the rainbow LED can be changed based on the condition(s). The light source(s) can be located inside or outside a bore of the MRI scanner.

In certain exemplary embodiments of the present disclosure, the condition(s) can include (i) a start of the MRI scan(s), (ii) an end of the MRI scan(s), (iii) an indication that the patient being scanned should not move, (iv) an indication that the patient being scanned can move, (v) an indication that the patient being scanned should hold their breath, (vi) an indication that the patient being scanned can release their breath, or (vii) an indication that a table of the MRI scanner is about to move. The light source(s) can include a plurality (e.g., at least four) light sources, such as, e.g., (i) a first light source configured to output a color red, (ii) a second light source configured to output a color green, (iii) a third light source configured to output a color yellow, and/or (iv) a fourth light source configured to output a color blue.

In another exemplary embodiment of the present disclosure is an apparatus for alerting a patient of a condition(s) of an MRI scan(s), which can include for example, an MRI scanner arrangement performing the MRI scan, and a light source(s) coupled to the MRI scanner arrangement, which is configured to alert the patient of the condition(s).

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figure(s) showing illustrative embodiments of the present disclosure, in which:

FIG. 1 is an illustration of an exemplary MRI machine which can include and/or utilize an exemplary system, method, computer-accessible medium and apparatus integrated therewith according to an exemplary embodiment of the present disclosure;

FIG. 2A is a diagram illustrating a single light source or LED that can change color according to an exemplary embodiment of the present disclosure;

FIG. 2B is a diagram of a group of light sources or LEDs, one for each exemplary color, according to an exemplary embodiment of the present disclosure;

FIG. 3A is a diagram the single light source or LED of FIG. 2A having a screen with written instructions according to an exemplary embodiment of the present disclosure;

FIG. 3B is a diagram of the group of the light source or LEDs of FIG. 2B, having a screen with written instructions according to an exemplary embodiment of the present disclosure;

FIG. 4 is a flow chart of an exemplary method for controlling a light source according to an exemplary embodiment of the present disclosure; and

FIG. 5 is an illustration of an exemplary block diagram of an exemplary system in accordance with certain exemplary embodiments of the present disclosure.

Throughout the drawing(s), the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

Accordingly, an exemplary system, method, computer-accessible medium and apparatus can be provided that can improve patient compliance during MRI scans and, thus, can lead to an increase in the reliability of MRI examinations by automatically adjusting the lighting (e.g., ambient lighting) in or around the MRI scanner depending on the current examination step, and depending on whether patient compliance is needed or not. Many MRI scanners can be equipped with ambient lighting of the magnet bore adjacent to the patient table. The ambient lighting can often be realized using white light-emitting diodes (“LEDs”), which can be switched ON and OFF, or switched to different brightness levels (e.g., OFF, Level 1, Level 2, Level 3, etc.) using a physical control at the MRI scanner, or using the software that controls the MRI system.

The white LEDs can be replaced by one or more color LEDs with freely-adjustable colors (e.g., a Rainbow LED, a color-changing LED or a color-adjustable LED), which can be controlled by the software of the MRI system, or by the technician. For example, a single LED can be used, which can change color depending on the notification. This hardware modification can result in, for example, only minimal additional costs, as these components can frequently be used in consumer electronics, and it can be expected that these components can be easily integrated into MRI systems. Additional ambient-lighting LEDs can be installed inside or outside the magnet bore to achieve sufficient brightness and visibility of the color for different patient positions (e.g., feet first, head down, etc.).

The light can be configured to be visible to the patient during the scan. For example, the light can be located inside the bore of the MRI machine or outside the bore of the MRI machine (e.g., attached to the MRI machine or detached from the MRI machine). The light can also be placed on a wall near the patient or on the ceiling. Placement of the light can be dependent on the ability of the patient to see the light while the patient is inside of the MRI machine (e.g., inside of the bore of the MRI machine).

The color of the ambient lighting can be changed whenever the examination step changes, or slightly before a change of the examination step, according to a fixed color scheme. For example, the lighting can be switched to (a) GREEN if no scan is performed and the patient can move, (b) YELLOW if a scan is about to be started within the next few seconds (e.g., within 2 seconds), and (c) RED if data is being acquired and the patient must not move. The adjustment of the color and brightness can be performed automatically by the MRI software, and/or may not need interaction by the technician. The lighting during breath-hold scans can additionally be animated (e.g., BLINKING) to urge the patient to suppress respiration. Furthermore, the lighting during the breath-hold-command phase can be animated (e.g., change of color or blinking frequency) to reflect the remaining time until the start of the data acquisition.

The color of the ambient lightning can additionally be changed, for example, to a BLUE color, if the patient table is about to be moved. The brightness of the lightning can additionally be increased to a higher level whenever the door of the examination room is about to be opened, in order to make the patient aware that the technician is coming into the examination room. The exemplary lighting can be changed depending on the level of patient activity during data acquisition. For example, if an imaging sequence with integrated motion assessment detects significant patient activity, the lighting can be animated (e.g., BLINKING) to urge the patient to stop the movement. An additional lighting color can be used during the acquisition of adjustment data (e.g., shimming and/or frequency adjustment), where suppression of motion can be beneficial, but not mandatory. To achieve compliance of patients unable to speak the same language as the technician, instruction cards in different languages can be provided that can explain the meaning of the different lighting colors, and can describe what the patient has to do during the individual phases of the MRI procedure.

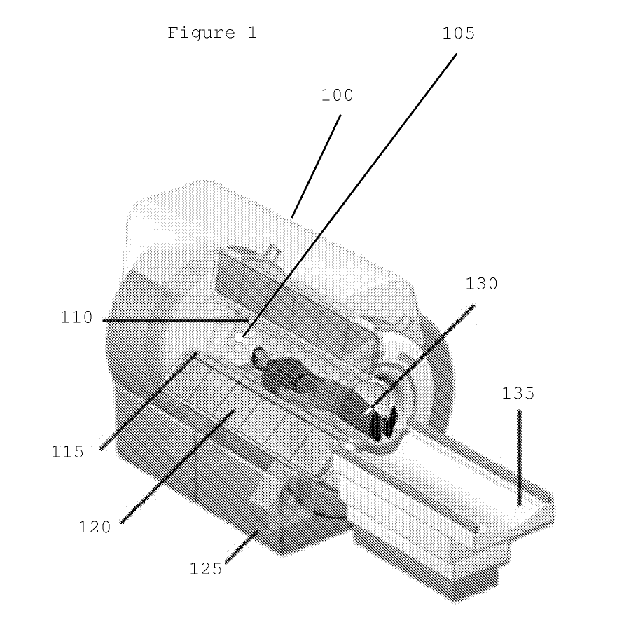

FIG. 1 illustrates an exemplary MRI machine 100 with an exemplary system, computer-accessible medium and apparatus 105 according to an exemplary embodiment of the present disclosure which can be provided or integrated therein. The MRI machine 100 can include a radio frequency coil 110, gradient coils 115, a magnet 120 and a scanner 125. The exemplary system, computer-accessible medium and apparatus 105 according to an exemplary embodiment of the present disclosure, all of which can utilize an exemplary method of the present disclosure, can be integrated at a position inside of the MRI machine such that a patient 130, lying on a patient table 135, can view the exemplary system, computer-accessible medium and apparatus 105 when the patient 130 is inside of the MRI machine 100.

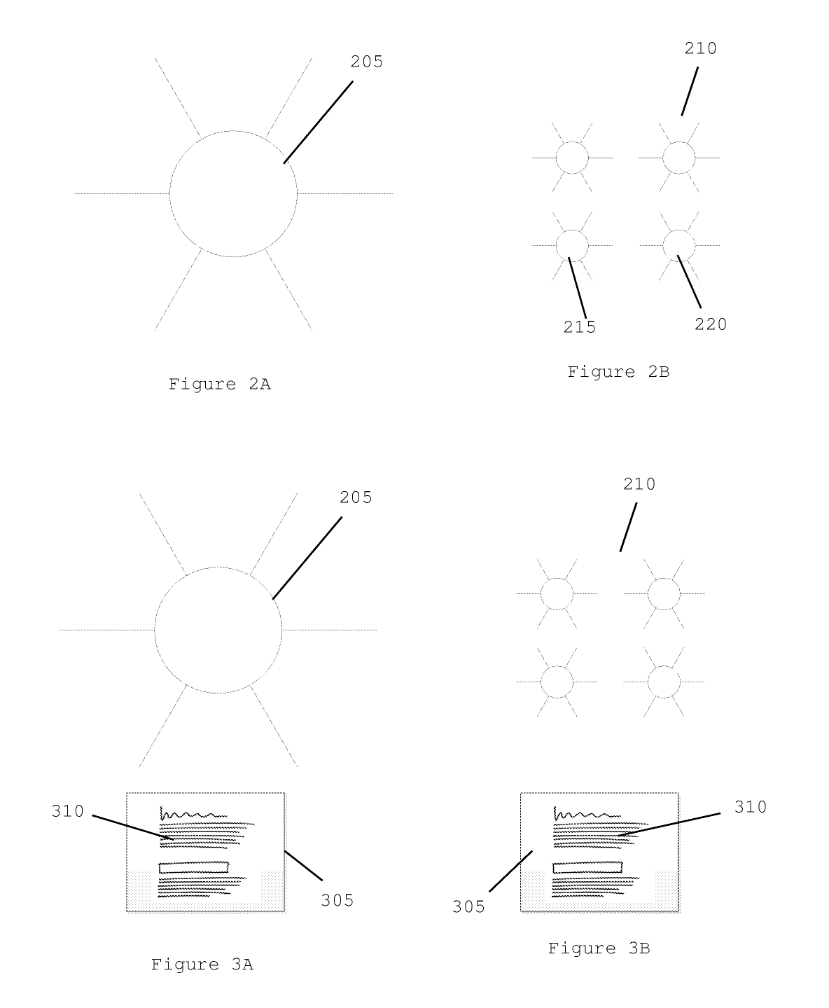

FIG. 2A shows an exemplary diagram of a light source or LED 205 used by and/or in the exemplary system, method, computer-accessible medium and apparatus. For example, the exemplary LED or light source 205 can be or include a multicolor LED that is capable or configured of changing colors depending on the voltage applied thereto. Thus, e.g., a single light can be used, and the color of the single light can change, as described herein, based on the indication being provided to the patient. Alternatively or in addition, as shown in FIG. 2B, the exemplary system, method, computer-accessible medium and apparatus can include and/or utilize a group of light sources or LED 210 (e.g., 2, 3, 4 or 5 light sources, etc.). Each light source can be configured to illuminate a single color only. Thus, for example, if the color red should be indicated, based on the colors described above, then the light source 215, which can only illuminate using the color red, can be used. If the color blue should be indicated, based on the colors described above, then only light source 220, which can only illuminate using the color blue, can be used. Additional lights can be used individually for the colors green, and yellow, etc.

In a further exemplary embodiment of the present disclosure, as shown in FIGS. 3A and 3B, in addition to either the single light source 205, or group of light sources 210, a screen 305 can be included at or near the light source(s). For example, the screen 305 can have written or printed instruction 310, which can correspond to the instructions being indicated by the light source(s). Thus, for example, if a red light is illuminated, which can indicate that data is being acquired and the patient must not move, instructions 310 on screen 305 can indicate to the patient that he or she should not move. The instructions 310 can then change depending on a further illumination of a different color of the lights. For example, the instructions 310 can be presented in different languages, depending on the language spoken by the patient.

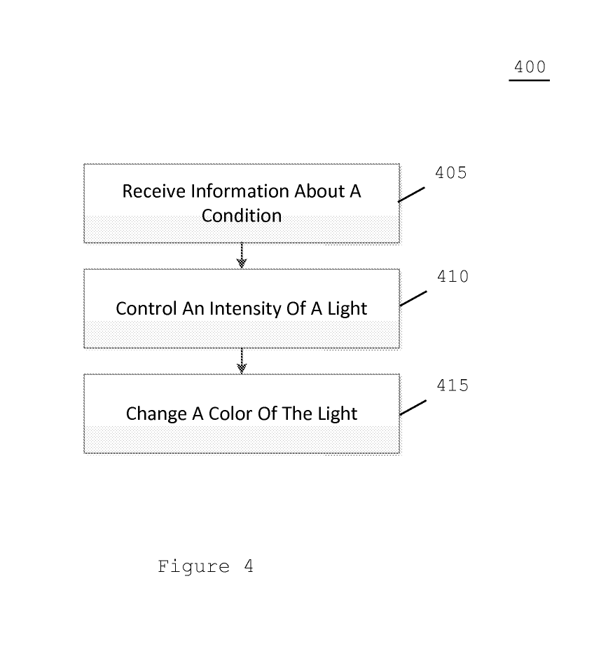

FIG. 4 shows a flow chart that illustrates a method 400 for controlling one or more light sources. For example, at procedure 405, information about a condition can be received, which can include (i) a start of the at least one MRI scan, (ii) an end of the at least one MRI scan, (iii) an indication that the patient being scanned should not move, (iv) an indication that the patient being scanned can move, (v) an indication that the patient being scanned should hold their breath, (vi) an indication that the patient being scanned can release their breath, and/or (vii) an indication that a table of the MRI scanner is about to move. At procedure 410, an intensity of the one or more lights can be controlled, and at procedure 415, a color of the one or more lights can be controlled.

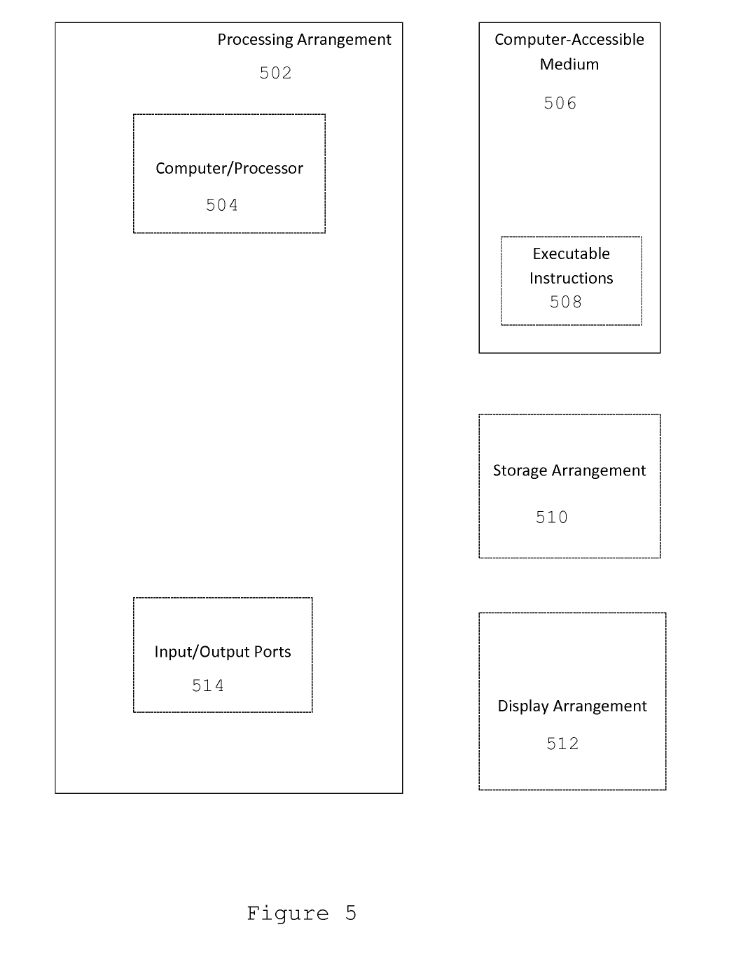

FIG. 5 shows a block diagram of an exemplary embodiment of a system according to the present disclosure. For example, exemplary procedures in accordance with the present disclosure described herein can be performed by a processing arrangement and/or a computing arrangement 502. Such processing/computing arrangement 502 can be, for example entirely or a part of, or include, but not limited to, a computer/processor 504 that can include, for example one or more microprocessors, and use instructions stored on a computer-accessible medium (e.g., RAM, ROM, hard drive, or other storage device).

As shown in FIG. 5, for example a computer-accessible medium 506 (e.g., as described herein above, a storage device such as a hard disk, floppy disk, memory stick, CD-ROM, RAM, ROM, etc., or a collection thereof) can be provided (e.g., in communication with the processing arrangement 502). The computer-accessible medium 506 can contain executable instructions 508 thereon. In addition or alternatively, a storage arrangement 510 can be provided separately from the computer-accessible medium 506, which can provide the instructions to the processing arrangement 502 so as to configure the processing arrangement to execute certain exemplary procedures, processes and methods, as described herein above, for example.

Further, the exemplary processing arrangement 502 can be provided with or include an input/output arrangement 514, which can include, for example a wired network, a wireless network, the internet, an intranet, a data collection probe, a sensor, etc. As shown in FIG. 5, the exemplary processing arrangement 502 can be in communication with an exemplary display arrangement 512, which, according to certain exemplary embodiments of the present disclosure, can be a touch-screen configured for inputting information to the processing arrangement in addition to outputting information from the processing arrangement, for example. Further, the exemplary display 512 and/or a storage arrangement 510 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. A non-transitory computer-accessible medium having stored thereon computer-executable instructions for alerting a patient of at least one condition during at least one magnetic resonance imaging (MRI) scan procedure, wherein, when a computer arrangement executes the instructions, the computer arrangement is configured to perform procedures comprising:
receiving information related to the at least one condition; and
controlling an intensity of at least one light source located near or on an MRI scanner based on the at least one condition.

2. The computer-accessible medium of claim 1, wherein the at least one light source includes at least one light-emitting diode (LED).

3. The computer-accessible medium of claim 2, wherein the LED is a white LED.

4. The computer-accessible medium of claim 2, wherein the LED is a rainbow LED.

5. The computer-accessible medium of claim 4, wherein the computer arrangement is further configured to change a color of the rainbow LED based on the at least one condition.

6. The computer-accessible medium of claim 1, wherein the at least one light source is located inside a bore of the MRI scanner.

7. The computer-accessible medium of claim 1, wherein the at least one light source is located outside a bore of the MRI scanner.

8. The computer-accessible medium of claim 1, wherein the at least one condition includes at least one of (i) a start of the at least one MRI scan, (ii) an end of the at least one MRI scan, (iii) an indication that the patient being scanned should not move, (iv) an indication that the patient being scanned can move, (v) an indication that the patient being scanned should hold their breath, (vi) an indication that the patient being scanned can release their breath, or (vii) an indication that a table of the MRI scanner is about to move.

9. The computer-accessible medium of claim 1, wherein the at least one light source comprises at least four light sources, wherein (i) a first one of the light sources is configured to output a color red, (ii) a second one of the light sources is configured to output a color green, (iii) a third one of the light sources is configured to output a color yellow, and (iv) a fourth one of the light sources is configured to output a color blue.

10. A method for alerting a patient of at least one condition of at least one magnetic resonance imaging (MRI) scan procedure, comprising:
receiving information related to the at least one condition; and
with a computer arrangement, controlling at least one light source located near or on an MRI scanner based on the at least one condition.

11. The method of claim 10, further comprising a computer hardware arrangement configured to cause the control of the at least one light source.

12. An apparatus for alerting a patient of at least one condition of at least one magnetic resonance imaging (MRI) scan, comprising:
an MRI scanner arrangement performing the MRI scan; and
at least one light source coupled to the MRI scanner arrangement, and configured to alert the patient of the at least one condition.

13. The apparatus of claim 12, wherein the at least one light source includes at least one light-emitting diode (LED).

14. The apparatus of claim 13, wherein the LED is a white LED.

15. The apparatus of claim 13 wherein the LED is a rainbow LED.

16. The apparatus of claim 15, wherein the rainbow LED is configured to change color based on the at least one condition.

17. The apparatus of claim 12, wherein the at least one light source is located inside a bore of the MRI scanner.

18. The apparatus of claim 12, wherein the at least one light source is located outside a bore of the MRI scanner.

19. The apparatus of claim 12, wherein the at least one condition includes at least one of (i) a start of the at least one MRI scan, (ii) an end of the at least one MRI scan, (iii) an indication that the patient being scanned should not move, (iv) an indication that the patient being scanned can move, (v) an indication that the patient being scanned should hold their breath, (vi) an indication that the patient being scanned can release their breath, or (vii) an indication that a table.

20. The apparatus of claim 12, wherein the at least one light source comprises at least four light sources, wherein (i) a first one of the light sources is configured to output a color red, (ii) a second one of the light sources is configured to output a color green, (iii) a third one of the light sources is configured to output a color yellow, and (iv) a fourth one of the light sources is configured to output a color blue.
